(19) United States
(12) Patent Application Publication
(10) Pub. No.: US 2026/0327401 A1
(43) Pub. Date: Oct. 22, 2026

(54) METHODS FOR DEPOLYMERIZING POLYAMIDES USING ORGANOLANTHANIDE CATALYSTS

(71) Applicant: Northwestern University, Evanston, IL (US)

(72) Inventors: Liwei Ye, Evanston, IL (US); Yosi Kratish, Evanston, IL (US); Tobin J. Marks, Evanston, IL (US)

(21) Appl. No.: 19/483,774

(22) PCT Filed: Apr. 25, 2024
(86) PCT No.: PCT/US2024/026137

§ 371 Date: Nov. 12, 2025

Related U.S. Application Data
(60) Provisional application No. 63/466,763, filed on May 16, 2023.

(51) Int. Cl. C08J 11/16; B01J 31/16; B01J 31/22; B29B 17/04; B29K 77/00; B29L 28/00; B29L 31/00; B29L 31/48; C07D 201/12

(52) U.S. Cl. CPC C08J 11/16; B01J 31/1608; B01J 31/2295; B29B 17/04; C07D 201/12; B01J 2531/36; B01J 2531/37; B01J 2531/38; B29B 2017/0496; B29K 2077/00; B29L 2028/00; B29L 2031/4842; B29L 2031/707; B29L 2031/7322; B29L 2031/753; C08J 2377/02

(57) ABSTRACT

Methods for depolymerizing a polyamide are provided which may comprise combining a polyamide and an organolanthanide catalyst to depolymerize the polyamide to a product, wherein the organolanthanide catalyst comprises a lanthanide metal, an active ligand bound to the lanthanide metal, and an ancillary ligand bound to the lanthanide metal, wherein the ancillary ligand is selected from conjugated carbocyclic groups and the active ligand is selected from those having a formula -EHₘ(XRₙ)ₒ, wherein E is selected from N, P, C, Si, Ge, and Sn; X is independently selected from H, N, P, C, Si, Ge, and Sn; and R is independently selected from H, alkyl, aryl, alkoxyl, silyl, germyl, and stannyl; wherein m is 0 or 1, n is 2 or 3, and o is 2 or 3.

### CROSS-REFERENCE TO RELATED APPLICATIONS

The present application claims priority to U.S. provisional patent application No. 63/466,763 that was filed May 16, 2023, the entire contents of which are incorporated herein by reference

### REFERENCE TO GOVERNMENT RIGHTS

This invention was made with government support under grant numbers DE-SC0022290 and DE-FG02-03ER15457 awarded by the Department of Energy, and grant number CHE1856619 awarded by the National Science Foundation. The government has certain rights in the invention.

### BACKGROUND

Catalysis is central to countless chemical transformations and holds great promise for the efficient recycling of commodity plastics. Plastics are ubiquitous, versatile, and low-cost polymeric materials that have dramatically enhanced the quality of human life for more than a century. Currently, plastics are being produced at an annual rate of 450 million tons, which is projected to double by 2045. Nevertheless, with only limited tools available to address plastics end-of-life, their accumulation has emerged as a global environmental challenge. For example, Nylons (or polyamides), which are produced on a scale exceeding 8.9 million tons annually, significantly contribute to the non-degradable plastic waste pollution in oceans and landfills, reflecting their superior chemical robustness and lack of effective recycling technologies. Indeed, it is estimated that by 2050, plastic waste will outweigh fish in the ocean, with Nylon-6 contributing ca. 10% of ocean plastic pollution as discarded/lost fishing nets (so-called “ghost nets”). This amounts to >600,000 tons of abandoned fishing nets annually. Polyamides are one of the most frequently encountered polymers found in the stomachs of marine animals, highlighting the persistent challenge of Nylon pollution in global marine ecosystems.

Since its discovery by Schlack in 1938, Nylon-6 has emerged as a versatile thermoplastic with widespread application in the automotive, packaging, carpet, and textile industries, as well as in fishing gear. Industrially, Nylon-6 is readily prepared from ε-caprolactam via water-assisted ring-opening polymerization or anionic ring-opening polymerization (ROP) using acylated caprolactam as initiator. Nylon-6 commercial products dominate the global Nylon market with more than 50% of the total revenue, and a market size projected to exceed $21 billion by 2026. Despite the attractions of Nylon-6, including high tensile strength, rigidity, toughness, as well as thermal and abrasion resistance, end-of-life management of Nylon waste falls far below the worldwide production rate.

### SUMMARY

Provided are methods for depolymerizing polyamides, e.g., Nylon-6, using certain organolanthanide catalysts which are further described below. The Example, below, describes experimental results demonstrating that mild conditions may be used in the methods (e.g., temperatures as low as 220° C. and reaction times of less than a few hours) along with catalyst loadings as low as 0.2 mol % to afford monomers of the polyamides, e.g., ¿-caprolactam, in surprisingly high yields (e.g., 100%). Moreover, the methods are not limited to virgin polyamide powders, but are compatible with a variety of post-consumer polyamide products, e.g., fishing nets, carpet fibers, clothing, medical gloves, etc. No solvents or other additives are required. The organolanthanide catalysts, e.g., in a reaction mixture with a polyamide, are also encompassed by the present disclosure.

In embodiments, a method for depolymerizing a polyamide comprises combining a polyamide and an organolanthanide catalyst to depolymerize the polyamide to a product, wherein the organolanthanide catalyst comprises a lanthanide metal, an active ligand bound to the lanthanide metal, and an ancillary ligand bound to the lanthanide metal, wherein the ancillary ligand is selected from conjugated carbocyclic groups and the active ligand is selected from those having a formula -EHₘ(XRₙ)ₒ, wherein E is selected from N, P, C, Si, Ge, and Sn; X is independently selected from H, N, P, C, Si, Ge, and Sn; and R is independently selected from H, alkyl, aryl, alkoxyl, silyl, germyl, and stannyl; wherein m is 0 or 1, n is 2 or 3, and o is 2 or 3.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A and 1B show existing catalytic approaches to Nylon-6 chemical recycling. By contrast, FIG. 1C shows a catalytic approach to Nylon-6 chemical recycling according to an illustrative embodiment of the present application.

FIG. 2A shows a reaction scheme for Nylon-6 depolymerization to ε-caprolactam using an illustrative embodiment of the present application. FIG. 2B shows structures of several illustrative catalysts that may be used with the disclosed methods.

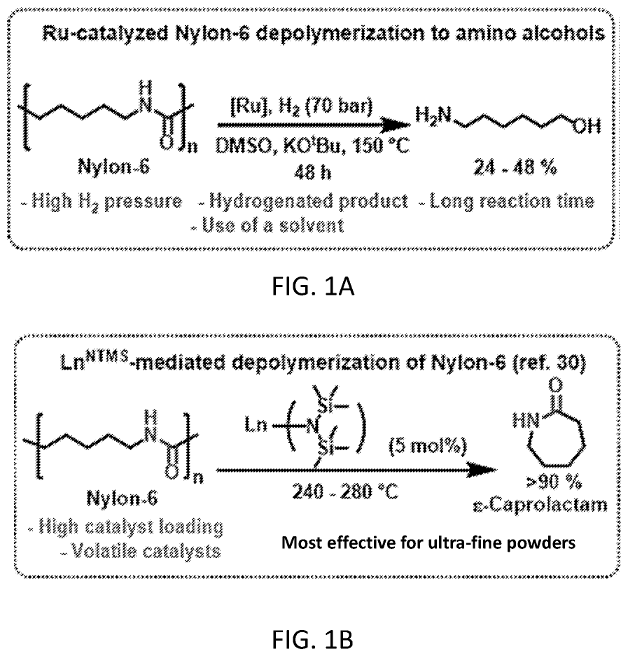

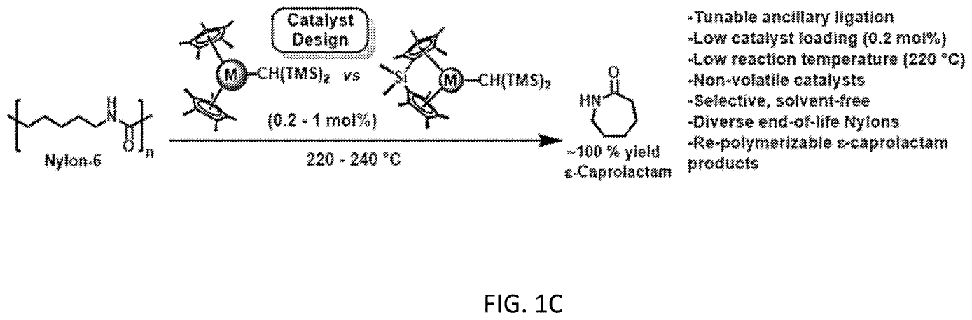

### DETAILED DESCRIPTION

Provided are methods for depolymerizing polyamides. In an embodiment, such a method comprises combining a polyamide and an organolanthanide catalyst under conditions to depolymerize the polyamide.

The polyamide to be depolymerized by the present methods is a polymer composed of monomers covalently bound into an extended chain via amide linking groups. The term “monomer” refers to the chemical reactant that is incorporated to form the extended chain and the amide linking groups during a polymerization reaction. The polyamide may be an aliphatic polyamide (i.e., not comprising aromatic rings) or an aromatic polyamide (i.e., comprising aromatic rings). In embodiments, the polyamide is an aliphatic polyamide. The polyamide may be a homopolymer (i.e., formed from a single type of monomer) or a heteropolymer (i.e., formed from more than one type of monomer, e.g., two; “heteropolymers” may be referred to as copolymers). The term “type” refers to a chemical formula such that a single type means the same chemical formula and different type means different chemical formulas.

The polyamide may be one which is formed by ring-opening polymerization of a cyclic amide (i.e., lactam). In such embodiments, the monomer is a cyclic amide and the polymerization reaction is ring-opening polymerization. Illustrative cyclic amides include 2-pyrrolidone, 2-piperidone, ¿-caprolactam, enantholactam, capryllactam, pelargolactam, azacycloundecan-2-one, and azacyclotridecan-2-one. As noted below, the polyamide may be one formed from a single type of cyclic amide or multiple, different types (e.g., 2 different types) of cyclic amides.

The polyamide may be one which is formed from condensation of an amine (including a diamine) with an acid (including a diacid). The amine and the acid may be provided on a single chemical compound such as 11-aminoundecanoic acid and ε-aminolauric acid. Alternatively, the amine and the acid may be provided on two different chemical compounds such as hexamethylene diamine and adipic acid. In these embodiments, the monomer is the single chemical compound (with the amine and acid groups) or the two chemical compounds (the diamine and the diacid), and the polymerization reaction is condensation.

The polyamide may be identified by reference to the monomers (e.g., cyclic amide, diamine, diacid) used to form the polyamide, recognizing that the chemical form of these monomers may be modified by the ensuing polymerization reaction that provides the amide linking groups of the polyamide.

Illustrative polyamides to be depolymerized in the present methods include poly(2-pyrrolidinone) (Nylon-4), poly(2-piperidone) (Nylon-5), poly(hexano-6-lactam) (Nylon-6), polyenanthamide (Nylon-7), polycapryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), and poly[imino (1,6-dioxo hexamethylene)imino hexamethylene] (Nylon-66). Other illustrative polyamides include copolymer polyamides formed from two or more different types of any of the cyclic amides described above. Other illustrative polyamides include copolymer polyamides formed from one of any of the cyclic amides described above and a strained cyclic amide, e.g., 6-azabicyclo[3.2.1]octan-7-one. Another illustrative polyamide is the copolymer Nylon-6/66. Another illustrative polyamide is the copolymer poly(ε-caprolactone-co-s-caprolactam).

The present methods may be used to depolymerize any of the disclosed polyamides. The depolymerization process deconstructs the polyamide into a product. In embodiments, the depolymerization process provides a monomer (e.g., a cyclic amide) from which the polyamide was formed, i.e., the product comprises (or consists of) the monomer of the polyamide. Throughout this disclosure, the terms “a monomer” and “monomer” encompass both a single type of monomer and multiple, different types of monomers, i.e., depending upon the particular polyamide. In such embodiments, this monomer may be recovered and used to reform the polyamide. This is by contrast to depolymerization processes which do not provide a monomer, but rather a product comprising a chemical compound which cannot reform the polyamide absent additional steps to convert the chemical compound to the monomer. However, the present methods encompass both types of depolymerization processes, i.e., those that provide the monomer of the polyamide and those that provide a different type of product.

In embodiments, the methods depolymerize Nylon-6 to produce ε-caprolactam. In embodiments, the methods depolymerize Nylon-4 to produce 2-pyrrolidone. In embodiments, the methods depolymerize Nylon-5 to produce 2-pyrrolidone. In embodiments, the methods depolymerize Nylon-7 to produce enantholactam. In embodiments, the methods depolymerize Nylon-8 to produce capryllactam. In embodiments, the methods depolymerize Nylon-9 to produce pelargolactam. In embodiments, the methods depolymerize Nylon-10 to produce azacycloundecan-2-one. In embodiments, the methods depolymerize Nylon-11 to produce azacyclotridecan-2-one. In embodiments, the methods depolymerize Nylon-12 to produce ω-aminolauric acid. For copolymer polyamides, the methods produce a mixture of the component monomers.

A single type of polyamide or multiple, different types of polyamides may be used in the present methods. The polyamide being depolymerized may comprise or consist of any of the disclosed polyamides or a combination thereof.

The organolanthanide catalysts used in the present methods are organometallic compounds comprising a lanthanide metal and ligands bound to the lanthanide metal, the ligands comprising at least one active ligand. One or more other ligands bound to the lanthanide metal may be ancillary ligands. An active ligand is of a different type, i.e., has a different chemical formula from an ancillary ligand. In embodiments, the organolanthanide catalyst comprises (or consists of) the lanthanide metal, a single active ligand bound to the lanthanide metal, and two (including not more than two) ancillary ligands bound to the lanthanide metal. The two ancillary ligands may be of the same type.

The lanthanide metal may be selected from Sc, Y, La, Ce, Pr, Nd, Pm, Sm, Gd, Tb, Dy, Ho, Er, Tm, Yb, Lu and mixtures thereof. (In the present disclosure, both Sc and Y may be considered to be lanthanide metals.) In embodiments, the lanthanide metal is La, Lu, Y, or Sm. In embodiments, the lanthanide metal is La, Y, or Sm. In embodiments, the lanthanide metal is Y.

The active ligand may have Formula I: -EHₘ(XRₙ)ₒ, wherein E is selected from N, P, C, Si, Ge, and Sn; each X is independently selected from H, N, P, C, Si, Ge, and Sn; each R is independently selected from H, alkyl, aryl, alkoxyl, silyl, germyl, and stannyl; wherein m is 0 or 1; n depends upon the selected X (e.g., n=2 or 3); and o depends upon the selected E and m (e.g., 0=2 or 3). In embodiments, E is N or P, m=0, o=2 (see Formula IA below). In embodiments, E is C, Si, Ge, or Sn, m=1, and o=2 (see Formula IB below). In embodiments, E is C, Si, Ge, or Sn (see Formula IC below). In Formula I (and all its related formulas herein, e.g., Formulas IA, IB, IC, etc.), the “-” denotes the covalent bond of the ligand to the lanthanide metal.

The active ligand may have Formula IA: -E(XRₙ)₂ wherein E is selected from N, and P; and X, R, and n are as defined above with respect to Formula I. In embodiments, the active ligand has Formula IA1: —N(XRₙ)₂, wherein X, R, and n are as defined above with respect to Formula I. In embodiments, the active ligand has Formula IA2: —N(SiRₙ)₂ wherein R and n are as defined above with respect to Formula I. In embodiments, the active ligand has Formula IA3: —N(SiRₙ)₂ wherein R is alkyl (e.g., a linear alkyl such as methyl) and n is 3.

The active ligand may have Formula IB: -EH(XRₙ)₂, wherein E is selected from C, Si, Ge, and Sn; and X, R, and n are as defined above with respect to Formula I. In embodiments, the active ligand has Formula IB1: —CH(XRₙ)₂ wherein X, R, and n are as defined above with respect to Formula I. In embodiments, the active ligand has Formula IB2: —CH(SiRₙ)₂ wherein R and n are as defined above with respect to Formula I. In embodiments, the active ligand has Formula IB₃: —CH(SiRₙ) 2 wherein R is alkyl (e.g., a linear alkyl such as methyl) and n is 3. Finally, it is noted that other active ligands having a similar basicity to —CH(Si(CH₃)₃)₂ may be used.

The active ligand may have Formula IC: -EHₘ(XRₙ)ₒ, wherein E is C, Si, Ge, or Sn and X, R, m, n, and o are as defined above with respect to Formula I. In embodiments, the active ligand has Formula IC1: —CHₘ(XRₙ)ₒ, wherein X, R, m, n, and o are as defined above with respect to Formula I. In embodiments, the active ligand has Formula IC2: —C(XRₙ)₃, wherein X, R, and n are as defined above with respect to Formula I. Since each X and R may be independently selected as noted above (which is the case for all formulas herein), Formula IC2 encompasses active ligands such as —C(XRₙ)₂(X′R′ₙ) and —C(XRₙ)₂(X′R′ₙ)(X″R″ₙ) (wherein X, X′, X″ are different from one another and R, R′, R″ are different from one another). However, Formula IC2 also encompasses active ligands in which each X and each R are the same.

As noted above, the ancillary ligand is a different chemical compound from the active ligand. Moreover, the ancillary ligand is a chemical compound other than those encompassed by Formula I and its related formulas. Suitable ancillary ligands may be selected from conjugated carbocyclic groups. There may be two such conjugated carbocyclic ancillary ligands such that the organolanthanide catalyst may be referred to as a lanthanocene catalyst. The conjugated carbocyclic groups being used as the ancillary ligands may be unsubstituted, monocyclic, or both. In other embodiments, substituted conjugated carbocyclic groups may be used. In other embodiments, polycyclic conjugated carbocyclic groups may be used. The meaning of “substituted” and “monocyclic” and “polycyclic” is analogous to that defined below for “aryl groups.” Illustrative conjugated carbocyclic groups include cyclopentadienyl and pentamethylcyclopentadienyl. In embodiments, two conjugated carbocyclic groups are used as the ancillary ligands and they are connected via a bridging group such that the lanthanocene catalyst may be referred to as an ansa-lanthanocene catalyst. Illustrative bridging groups include alkyl groups and silyl groups, e.g., —Si(CH₃)₂—. (The “-” connotes the bonds to the respective ancillary ligands.)

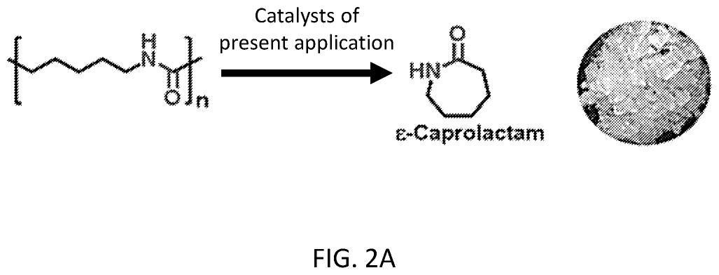

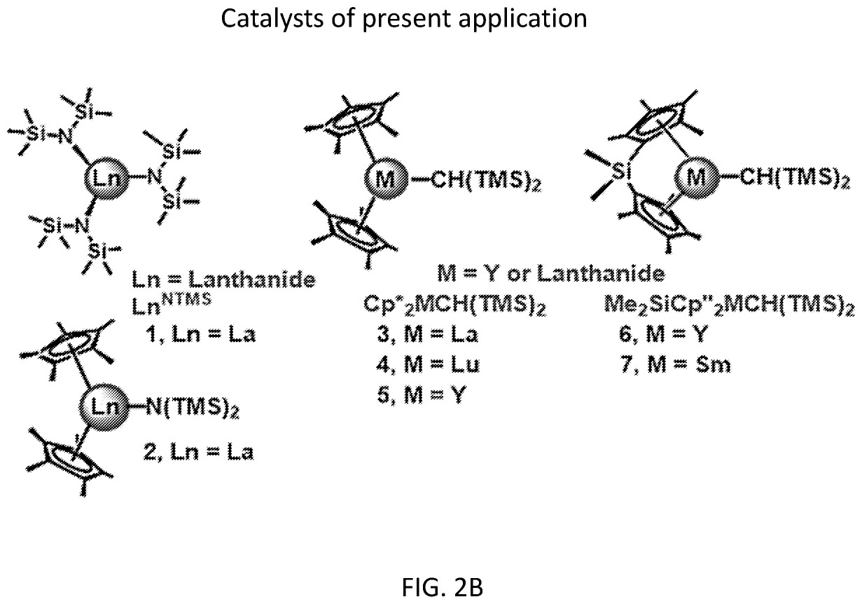

Organolanthanide catalysts are encompassed which are based on various combinations of the lanthanide metals, active ligands, and ancillary ligands described above. However, illustrative organolanthanide catalysts are shown in FIG. 2B. In embodiments, the organolanthanide catalyst is selected from Cp*₂LnCH(TMS)₂(Cp*=η⁵−C₅Me₅, Ln=the lanthanide metal, TMS=SiMe₃)) and Me₂SiCp″₂LnCH(TMS)₂(Cp″=η⁵—C₅Me₄). In some such embodiments, Ln is La, Y, or Sm. In some such embodiments, Ln is Y. Herein, η⁵ means that the metal atom is bonded to the five carbon atoms of each of the conjugated carbocyclic groups.

A single type of organolanthanide catalyst or multiple, different types of organolanthanide catalysts may be used. The organolanthanide catalyst being used to catalyze depolymerization may comprise or consist of any of the disclosed organolanthanide catalysts or a combination thereof.

Regarding the alkyl groups described herein, “alkyl group” refers to a linear, branched, or cyclic alkyl group in which the number of carbons may range from, e.g., 1 to 8, 1 to 6, 1 to 4, 1 to 3, or 1 to 2. A cyclic alkyl group may be referred to as a cycloalkyl group. The alkyl group may be unsubstituted, by which it is meant the alkyl group contains no heteroatoms. An unsubstituted alkyl group encompasses an alkyl group in which one or more bonds to a carbon(s) or hydrogen(s) are replaced by a bond to an unsubstituted aromatic ring, e.g. benzyl. The alkyl group may be substituted, by which it is meant an unsubstituted alkyl group in which one or more bonds to a carbon(s) or hydrogen(s) are replaced by a bond to non-hydrogen and non-carbon atoms.

Regarding the aryl groups described herein, “aryl group” refers to a monocyclic aryl group having one aromatic ring (e.g., benzene) or a polycyclic group having more than one aromatic ring (e.g., two, three, etc. rings). Monocyclic aryl groups may be unsubstituted or substituted as described above with respect to alkyl groups. Regarding polycyclic groups, neighboring aromatic rings may be fused or unfused. The aromatic rings of a polycyclic group may be unsubstituted or substituted as described above with respect to monocyclic aryl groups.

Regarding “alkoxy” described herein, this term refers to —OR, wherein R is selected from alkyl (as defined herein) and “-” denotes the covalent bond, e.g., to X in Formula I and all its related formulas.

Regarding “silyl” described herein, this term refers to —SiR₃, wherein R is independently selected from H, alkyl, and aryl (each of which has been defined herein) and “-” denotes the covalent bond, e.g., to X in Formula I and all its related formulas.

Regarding “germyl” described herein, this term refers to —GeR₃, wherein R is independently selected from H, alkyl, and aryl (each of which has been defined herein) and “-” denotes the covalent bond, e.g., to X in Formula I and all its related formulas.

Regarding “stannyl” described herein, this term refers to —SnR₃, wherein R is independently selected from H, alkyl, and aryl (each of which has been defined herein) and “-” denotes the covalent bond, e.g., to X in Formula I and all its related formulas.

Regarding substituents in the groups described herein, including those of Formula I and all its related formulas, (as opposed to unsubstituted groups), non-hydrogen and non-carbon atoms include, e.g., halogen; oxygen; sulfur; nitrogen; phosphorus; silicon; germanium; and tin. In embodiments, the group, i.e., alkyl, aryl, alkoxy, silyl, germyl, stannyl, is unsubstituted (which does not preclude the presence of the O in alkoxy, the Si in silyl, the Ge in germyl, and the Sn in stannyl).

The conditions being used in the present methods to achieve depolymerization may refer to parameters such as the temperature, period of time, atmosphere, and organolanthanide catalyst loading. As the present methods may be carried out using a variety types of reactor systems, including batch reactor systems, semi-batch reactor systems, and continuous flow reactor systems, the conditions may also refer to a specific type of reactor system. Conditions may be selected in order to promote depolymerization and thus, to achieve a desired (e.g., maximum) yield of product (e.g., monomer). However, as noted above, the present methods are able to achieve high yields even using mild conditions and minimal catalyst loadings.

Regarding temperature, the temperature may be within 30° C., within 20° C., or within 10° C. of the melting temperature of the polyamide. The temperature may be above the melting temperature of the polyamide such that it is in its molten (liquid state) during the depolymerization. In embodiments, the temperature is no more than 300° C., no more than 275° C., no more than 265° C., no more than 255° C., or no more than 245° C. This includes a range of from 200° C. to 300° C., from 200° C. to 275° C. and from 200° C. to 250° C.

Regarding time, this may refer to a total period of time over which the polyamide and the organolanthanide catalyst are subjected to depolymerization. In embodiments, the period of time is less than 48 hours, less than 36 hours, less than 24 hours, or less than 10 hours. This includes a range of between any of these values, as well as from 1 minute to 1 hour, from 1 hour to 10 hours, and from 1 hour to 5 hours. For continuous flow reactor systems, flow rate, rather than time is a relevant parameter. In embodiments, the flow rate is from 5 sccm to 1500 sccm, although higher flow rates may be used. Reactor volume is another relevant parameter for continuous flow reactor systems. In embodiments, the reactor volume is from 50 mL to 1000 L, although greater reactor volumes may be used.

Regarding atmosphere, a vacuum may be used, e.g., 10⁻³ Torr or less. This includes a vacuum under 10⁻¹⁰ Torr. However, the method may be carried out at higher pressures, including in a range of from 10⁻² Torr to 1 atm. The method may be carried out under an inert atmosphere (e.g., N₂, dry air, argon).

Regarding organolanthanide catalyst loading, the loading may be no more than 10 mol %, no more than 8 mol %, or no more than 6 mol %. This includes a range between any of these values, as well as from 0.01 mol % to 5 mol %, from 0.01 mol % to 2 mol %, and from 0.01 mol % to 0.1 mol %. Mol % is calculated based on the of repeating unit of the polyamide. For example, if 1 g of Nylon-6 is used, the number of mol of repeating unit=0.00885 mol (repeating unit of Nylon-6 is 113 g/mol). Thus, the amount of catalyst used is 0.00885 mol*x mol %. It is noted that lower values of catalyst loading such as those described above may be achieved when using continuous reactor systems, as demonstrated in the Example below.

The present methods are generally carried out without using any liquid medium, e.g., solvent, for the organolanthanide catalyst and the polyamide. As such, the method may be referred to as being “solvent-free” and the like.

The polyamide (the specific chemical type of which has been described above) may be “virgin” polyamide, which generally refers to pure, as-synthesized polyamide that has not been further processed for use in a particular application (whether the virgin polyamide has been used or not). Alternatively, the polyamide may be “post-consumer” polyamide, which generally refers to a polyamide derived from a consumer product (whether that consumer product has actually been used or not). Post-consumer polyamide may include other components (e.g., other non-polyamide polymers such as polyolefins, polyesters) and may have been processed for use in a particular application (e.g., yarns, fishing nets, carpet fibers, clothing, medical gloves). In either embodiment, the morphology of the polyamide is not particularly limited. For example, virgin polyamide may be in the form of a powder, including powder composed of micron-sized particles. However, as demonstrated in the Example, below, the present methods achieve surprisingly high monomer yields from post-consumer polyamide that has been chopped into pieces significantly larger than the particles of virgin polyamide powder. In other words, no milling, e.g., cryogenic milling, is required to achieve high monomer yields from post-consumer polyamide using the present methods. Regardless of the source or morphology of the polyamide, prior to use in the methods, the polyamide may be washed and dried by heating under vacuum as described in the Example, below.

When combined for carrying out the present methods, the polyamide and the organolanthanide catalyst may be considered to form a reaction mixture comprising each of these components. As noted above, the reaction mixture need not comprise any liquid medium (e.g., solvent). (This does not preclude the presence of a liquid in the reaction mixture due to the use of molten polyamide.) Similarly, the reaction mixture need not comprise other additives. Thus, the reaction mixture may be characterized as being free of a liquid medium (other than the polyamide if in its molten state) and free of an additive. This includes the reaction mixture being free of one or more of the following: an ionic liquid; water or steam; ammonia; N,N-dimethylaminopyridine; acetic anhydride; an amine; a phosphoric acid (or salt thereof); a boric acid (or salt thereof); a sulfonic acid (or salt thereof); a carboxylic acid (or salt thereof); a carbonate; an alkali or alkaline earth oxide; an alkali or alkaline earth hydroxide; an alkali or alkaline earth carbonate; an alcohol. In embodiments, the reaction mixture consists of the polyamide and the organolanthanide catalyst. The embodiments in this paragraph do not preclude the ultimate presence of the monomer provided by the depolymerization, unreacted polyamide and/or other depolymerized product (e.g., polyamide fragments) in the reaction mixture. These embodiments also do not preclude the presence of components or impurities which may be inherently present in the reaction mixture due to the particular synthetic technique used to form the polyamide (e.g., virgin polyamide). These embodiments also do not preclude the presence of components which may be inherently present in the reaction mixture due to the source of the polyamide (e.g., post-consumer polyamide).

The present methods may further comprise recovering and/or recycling the organolanthanide catalysts from the reaction mixture. No liquid medium is required for recovery of the organolanthanide catalysts, which may be recovered by physically removing the catalysts from the reaction chamber. The recovered organolanthanide catalysts may be used to carry out the method one or more additional times (i.e., they may be recycled). Similarly, the present methods may further comprise recovering the depolymerized product, e.g., monomer, from the reaction mixture. No liquid medium is required for recovery which may be by vaporization or sublimation. Recovered product may be used for any desired purpose, e.g., recovered monomer may be used to synthesizing a new polymer, including a new polyamide, including the same type of polyamide that was depolymerized.

The present methods may be characterized by a yield of monomer. The yield is reported as (weight of monomer)/(starting weight of polyamide)*100%. The yield may be determined using ¹H NMR as described in the Example below. The yield may be an initial yield obtained by using fresh (i.e., unused) organolanthanide catalyst. The initial yield may be at least 70%, at least 75%, at least 80%, at least 85%, at least 90%, at least 95%, or at least 98%. This includes a range between any of these values, as well as from 70% to 100%, from 80% to 100%, and from 80% to 95%. The yield may be a yield obtained by using recovered organolanthanide catalyst which has been used one or more times (e.g., 1, 2, 3, 4, etc.). The yield from a recycled/recovered organolanthanide catalyst may be within ±20%, ±10%, ±5%, or ±2% of the initial yield. Any of the yield values in this paragraph may refer to a specific polyamide (e.g., Nylon-6), a specific monomer (e.g., ε-caprolactam), a specific organolanthanide catalyst (e.g., Cp*₂LaCH(TMS)₂ or Me₂SiCp″₂YCH(TMS)₂) and the method carried out under a specific set of conditions (e.g., batch reactor, temperature (e.g., 220° C. or 240° C.), time (e.g., 2 or 4 hours), under vacuum or an inert atmosphere of 1 atm, and loading (e.g., 0.2 or 1 mol %).

As noted above, the polyamide to be depolymerized by the present methods may be provided in a blend with non-polyamide polymers, e.g., a polyolefin (e.g., polyethylene, polypropylene) or a polyester (e.g., polyethylene terephthalate). The method may depolymerize the polyamide while leaving the other polymer intact. The term “intact” does not necessarily mean perfectly intact as a minor amount of the non-polyamide polymer may be decomposed. Thus, the present methods allow for separation of a polyamide from such a blend.

The present disclosure encompasses any of the organolanthanide catalysts described herein as well as reaction mixtures comprising (or consisting of) such catalysts with any of the disclosed polyamides.

### Example

### Introduction

Rational tailoring of catalytic systems offers highly desirable transformations targeting the growing environmental challenges associated with plastics pollution. For example, the identification of efficient catalysts to address alarming end-of-life Nylon pollution has not been sufficiently explored. Nylon-6 is a non-biodegradable high-performance engineering plastic with centuries chemical persistence, resulting in millions of tons of waste accumulation. This Example reports certain manipulations of an organolanthanide catalyst structure to achieve a highly efficient, solventless, and scalable Nylon-6 depolymerization process, affording monomer ε-caprolactam in up to 100% yield. Specifically, catalyst Cp*₂LaCH(TMS)₂ (Cp*=η⁵—C₅Me₅, TMS=SiMe₃) operates at catalyst loadings as low as 0.2 mol % and temperatures as low as 220° C. (See catalyst 3 in FIG. 2B.) For efficient deconstruction of more recalcitrant commodity Nylon-6 end-of-life articles such as fishing nets, carpets, and clothing, the robust and thermally stable ansa-metallocene catalyst Me₂SiCp″₂YCH(TMS)₂, (Cp″=η⁵—C₅Me₄) effects 100% conversion of these items into ε-caprolactam. (See catalyst 6 in FIG. 2B.) The collected product can be readily re-polymerized to afford pristine Nylon-6 with comparable molecular masses and structural regularity, providing an upcycling pathway for end-of-life Nylon plastics. Experimental/mechanistic studies supported by density functional theory (DFT) analyses reveal a “chain-hopping” depolymerization pathway for these catalysts.

### Experimental

### General Methods and Materials

Methods: All manipulations of catalyst synthesis were carried out with the exclusion of O₂ and moisture in oven-dried Schlenk-type glassware on either a dual-manifold Schlenk line, interfaced to a high-vacuum manifold (10⁻⁶ Torr), or in an Argon-filled MBraun glovebox with a high-capacity recirculatory (<1 ppm O₂). All depolymerization reactions were assembled by mixing the polymer and the appropriate catalyst in a N₂-filled MBraun glovebox in cylindrical 50 mL Schlenk tubes. Heating was supplied by a customized aluminum heating block with a fitted hole for the Schlenk tubes.

General materials: La(N (TMS) 2) 3, La^{NTMS} was obtained from Strem Chemicals. Cp*₂LaCH(TMS)₂, Cp*₂LuCH(TMS)₂, Cp*₂YCH(TMS)₂, Cp*₂LaN(TMS)₂, [Cp*₂LaH]₂, Me₂SiCp″²YCH(TMS)², were synthesized. (Jeske, G., et al. J. Am. Chem. Soc. 1985, 107 (26), 8091-8103; Den Haan, K. H., et al. Organometallics 1986, 5 (8), 1726-1733; and Jeske, G., et al., J. Am. Chem. Soc. 1985, 107 (26), 8103-8110.) All polymers were washed with 1 M KOH solution overnight, filtered and washed with H₂O, and dried under a high vacuum at 100° C. for at least 24 h prior to use. Pristine Nylon-6 powder with a mean particle size of 15-20 μm and a molecular weight of 11930 g/mol (as determined by GPC) was obtained from Goodfellow Inc.

Post-consumer plastics: All post-consumer products were chopped with a scissor into smaller pieces to fit into Schlenk tubes prior to depolymerization reactions. End-of-life commercial fishing net (minimum 2 years of usage) was purchased from a local fishing store. A sample of carpet was acquired from The Home Depot Inc. Nylon yarn was acquired from The Singer Company. A Nylon T-shirt was acquired from Walmart Inc. Medical gloves (containing 31% of Nylon-6) were acquired from Amazon Inc.

Nylon content determination method for medical gloves: in a 25 mL vial equipped with a stir bar, 1 g of scissor-cut medical gloves and 20 mL of hexafluoroisopropanol (HFIP) were added and allowed to stir overnight. The mixture was then filtered, and the solid residue was washed with 20 mL of HFIP and subsequently 40 mL of acetone. The HFIP filtrate was concentrated and added a few drops of CDCl₃ and loaded in an NMR tube for ¹H-NMR analysis. Nylon-6 was confirmed as the only HFIP-soluble plastic. The solid residue was washed with acetone, dried overnight and weighed (690 mg, 69% of 1 g sample). Nylon-6 content in these medical gloves was therefore determined to be 31%.

### Physical and Analytical Methods

NMR spectra were recorded on a Varian Bruker Avance III HD system equipped with a TXO Prodigy probe (500 MHz) spectrometer. Chemical shifts (δ) for ¹H-NMR were referenced to the internal solvent. Gel permeation chromatography (GPC) was used to analyze molecular weight and molecular weight dispersity indices of synthesized Nylon-6. An Agilent Infinity II 1260 high-performance liquid chromatography (HPLC) system was coupled with Wyatt MiniDAWN TREOS Multi-Angle Light Scattering (MALS, 3 angles) and Refractive Index (RI, 658 nm) detectors. Polymer concentrations of ~5.0 mg/mL in hexafluoroisopropanol (HFIP, ChemImpex) were prepared by dissolving polymer at 30° C. in a shaker, followed by filtration through a 0.2 μm syringe filter. Samples were injected at a volume of 100 μL into the system at a flow rate of 0.4 mL/min (HFIP with 20 mM NaTFA) through the column at 40° C., which consisted of 3 Plgel-HFIP columns in series with a guard column. Astra software was used to determine absolute Mₙ, M_{w}, and dispersity (Ð, M_{w}/Mₙ) using a dn/dc value of 0.241 mL/g (value found on American Polymer Standard Corporation website for nylon-6,6 in HFIP with 10 mM NaTFA). Polymethyl methacrylate and poly(ethylene terephthalate) standards were used to check instrumentation and validate results.

### General Depolymerization Procedure

General Procedure for depolymerization reactions. In a glove box, a 50 mL oven-dried Schlenk tube was charged with a magnetic stir bar, pristine Nylon-6 fine powder (or post-consumer Nylon product), and finely ground catalyst. The vessel was sealed tightly, and the polymer and catalyst were thoroughly mixed by stirring at room temperature for approximately 5 min. The Schlenk tube was then evacuated to 10⁻³ Torr, sealed, and heated to the specified temperature with slow magnetic stirring (50-100 rpm) for the specified time. Reaction time was recorded starting 2.5 min after the reaction tube was placed in the heating block, since it takes an average of 2.5 min for the reaction to reach the depolymerization temperature and the polymer to begin melting. During the reaction, the products sublime from the hot reaction zone and deposit as a crystalline layer on the cold wall of the reactor. After cooling to room temperature, the soluble part of the reaction mixture was dissolved in 3-4 mL of deuterated solvent, and mesitylene was added as an internal standard. A sample of this solution was withdrawn for NMR analysis. Yields were determined by 1H-NMR, comparing the signal integrals of ε-caprolactam and mesitylene.

### Procedures and NMR Spectra for Reactions in Table 1

General note: NMR spectra with the following signals labeled may be found in U.S. Ser. No. 63/466,763, filed May 16, 2023, which is incorporated herein by reference: deuterated solvent, the internal standard mesitylene, ε-caprolactam, the cyclic dimer/oligomers and open-chain amide, and the protonolysis products (such as Cp*H) of the selected catalysts. The cyclic dimer/oligomers, and the open chain amide were already present in the commercial Nylon-6 starting material and in some post-consumer products.

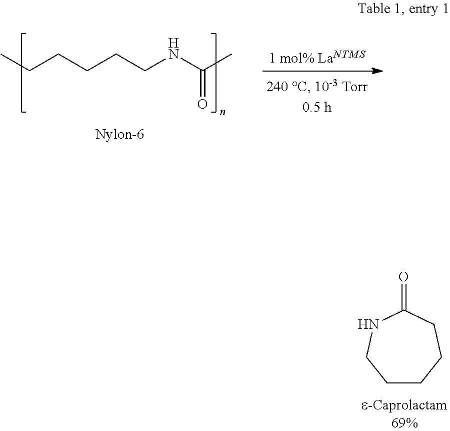

Exactly 100 mg of Nylon-6 pristine powder (0.89 mmol) and 5.5 mg of La^{NTMS} (8.9 μmol, 1 mol %) were reacted according to the general procedure, heating the reaction mixture to 240° C. for 0.5 h. Exactly 18.1 mg of mesitylene (0.150 mmol) was used as an internal standard. ε-caprolactam was obtained in 69% yield.

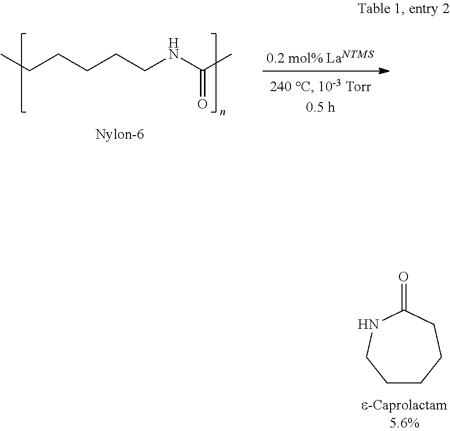

Exactly 100 mg of Nylon-6 pristine powder (0.89 mmol) and 1.1 mg of La^{NTMS} (1.8 μmol, 0.2 mol %) were reacted according to the general procedure, heating the reaction mixture to 240° C. for 0.5 h. Exactly 21.9 mg of mesitylene (0.181 mmol) was used as an internal standard. ε-caprolactam was obtained in 5.6% yield.

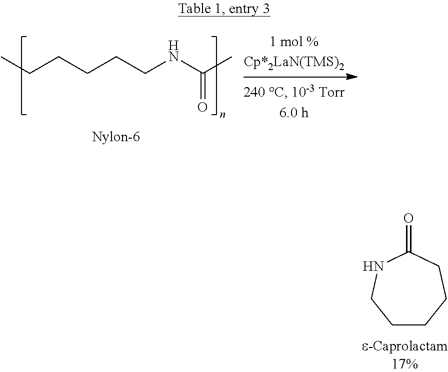

104 mg of Nylon-6 pristine powder (0.89 mmol) and 1.1 mg of Cp*₂LaN(TMS)₂ (8.9 μmol, 1 mol %) were reacted according to the general procedure, heating the reaction mixture to 240° C. for 6.0 h. Exactly 27.7 mg of mesitylene (0.230 mmol) was used as an internal standard. ¿-caprolactam was obtained in 95% yield.

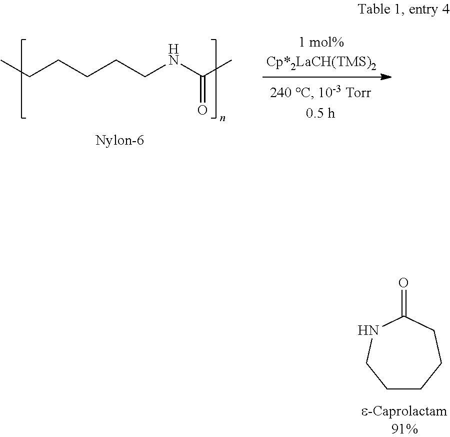

100.2 mg of Nylon-6 pristine powder (0.89 mmol) and 5.3 mg of Cp*₂LaCH(TMS)₂ (8.9 μmol, 1 mol %) were reacted according to the general procedure, heating the reaction mixture to 240° C. for 0.5 h. Exactly 21.0 mg of mesitylene (0.174 mmol) was used as an internal standard. ¿-caprolactam was obtained in 91% yield.

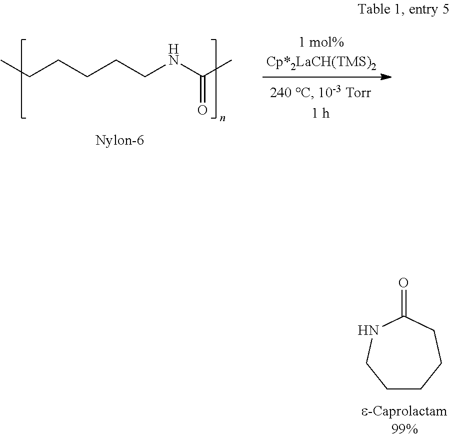

Exactly 100.0 mg of Nylon-6 pristine powder (0.89 mmol) and 5.3 mg of Cp*₂LaCH(TMS)₂ (8.9 μmol, 1 mol %) were reacted according to the general procedure, heating the reaction mixture to 240° C. for 1 h. Exactly 20.5 mg of mesitylene (0.170 mmol) was used as an internal standard. ε-caprolactam was obtained in 99% yield.

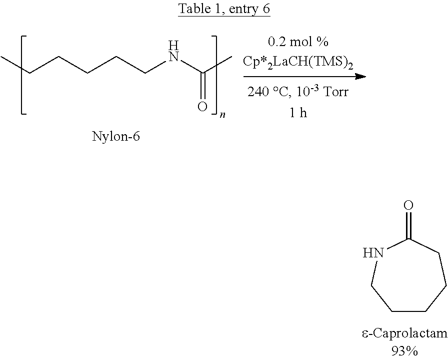

Exactly 100.0 mg of Nylon-6 pristine powder (0.89 mmol) and 1.1 mg of Cp*₂LaCH(TMS)₂ (1.8 μmol, 0.2 mol %) were reacted according to the general procedure, heating the reaction mixture to 240° C. for 1 h. Exactly 21.7 mg of mesitylene (0.180 mmol) was used as an internal standard. ε-caprolactam was obtained in 93% yield.

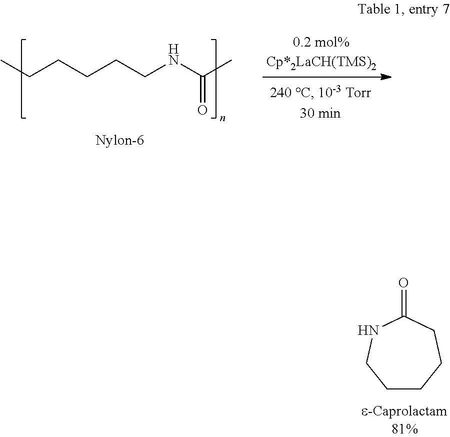

Exactly 100.0 mg of Nylon-6 pristine powder (0.89 mmol) and 1.1 mg of Cp*₂LaCH(TMS)₂ (1.8 μmol, 0.2 mol %) were reacted according to the general procedure, heating the reaction mixture to 240° C. for 30 minutes. Exactly 18.9 mg of mesitylene (0.157 mmol) was used as an internal standard. ¿-caprolactam was obtained in 81% yield.

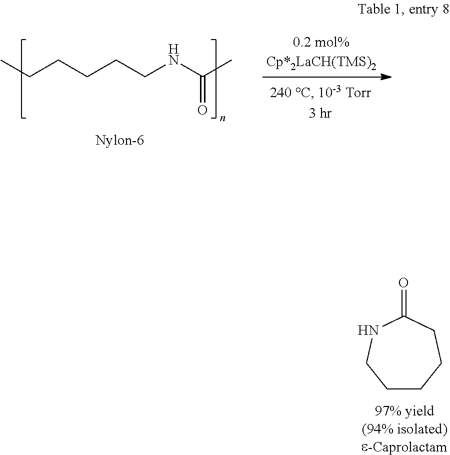

Exactly 1.045 g of Nylon-6 pristine powder (8.9 mmol) and 11 mg of Cp*₂LaCH(TMS)₂ (18 μmol, 0.2 mol %) were reacted according to the general procedure (except that a 100 mL Schlenk tube was used), heating the reaction mixture to 240° C. for 3 h. Exactly 149.6 mg of mesitylene (1.24 mmol) was used as an internal standard and was added along with ~10 mL of CDCl₃. ε-caprolactam was obtained in 97% yield based on 1H-NMR analysis (see below). The CDCl₃ solution mixture was then combined and evaporated to dryness. Crude s-caprolactam was recrystallized from 10 mL of hexane/acetone (3:1) solvent mixture over 2 days and was then filtered, washed with cold hexane and dried under high vacuum. Isolated white crystals (943 mg) were obtained with a yield of 94%.

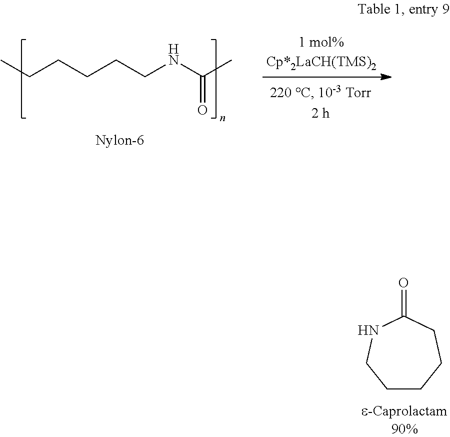

Exactly 99.8 mg of Nylon-6 pristine powder (0.89 mmol) and 5.3 mg of Cp*₂LaCH(TMS)₂ (8.9 μmol, 1 mol %) were reacted according to the general procedure, heating the reaction mixture to 220° C. for 2 h. Exactly 26.6 mg of mesitylene (0.221 mmol) was used as an internal standard. ε-caprolactam was obtained in 90% yield.

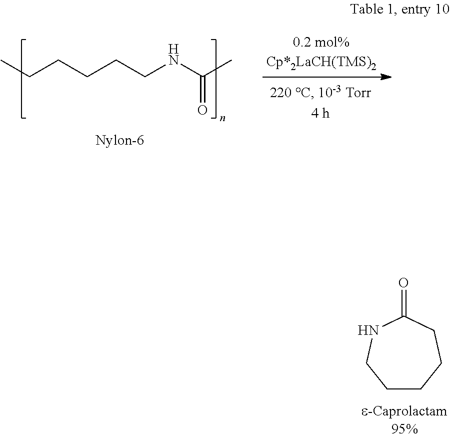

Exactly 100.0 mg of Nylon-6 pristine powder (0.89 mmol) and 1.1 mg of Cp*₂LaCH(TMS)₂ (1.8 μmol, 0.2 mol %) were reacted according to the general procedure, heating the reaction mixture to 220° C. for 4 h. Exactly 27.7 mg of mesitylene (0.230 mmol) was used as an internal standard. ¿-caprolactam was obtained in 95% yield.

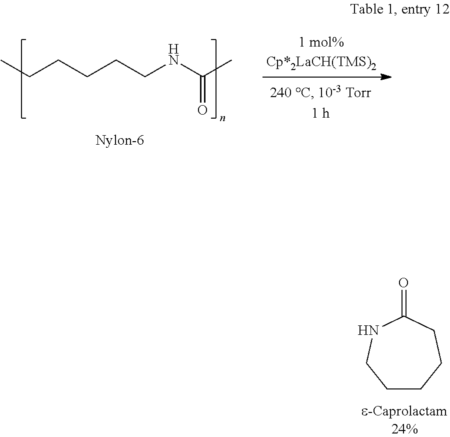

Exactly 100.0 mg of Nylon-6 pristine powder (0.89 mmol) and 5.2 mg of Cp*₂LuCH(TMS)₂ (8.9 μmol, 1 mol %) were reacted according to the general procedure, heating the reaction mixture to 240° C. for 1 h. Exactly 25.3 mg of mesitylene (0.211 mmol) was used as an internal standard. ε-caprolactam was obtained in 24% yield.

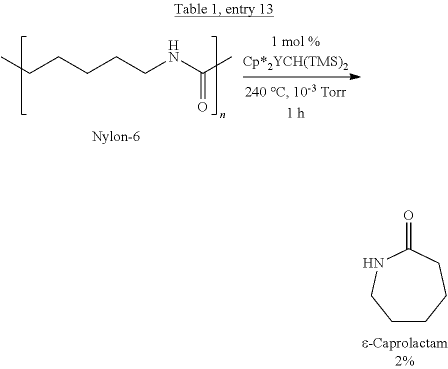

Exactly 100.0 mg of Nylon-6 pristine powder (0.89 mmol) and 4.9 mg of Cp*₂YCH(TMS)₂ (8.9 μmol, 1 mol %) were reacted according to the general procedure, heating the reaction mixture to 240° C. for 1 h. Exactly 22.8 mg of mesitylene (0.190 mmol) was used as an internal standard. ¿-caprolactam was obtained in 2% yield.

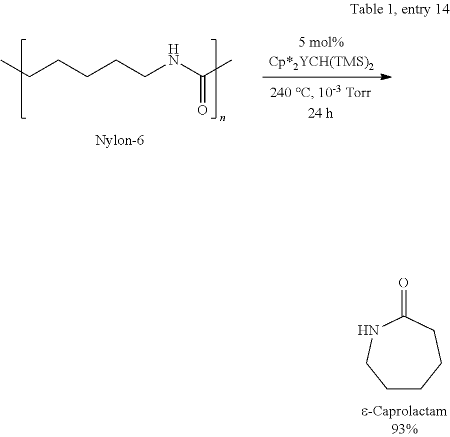

Exactly 97.0 mg of Nylon-6 pristine powder (0.88 mmol) and 24.5 mg of Cp*₂YCH(TMS)₂ (44.5 μmol, 5 mol %) were reacted according to the general procedure, heating the reaction mixture to 240° C. for 24 h. Exactly 15.2 mg of mesitylene (0.127 mmol) was used as an internal standard. ε-caprolactam was obtained in 93% yield.

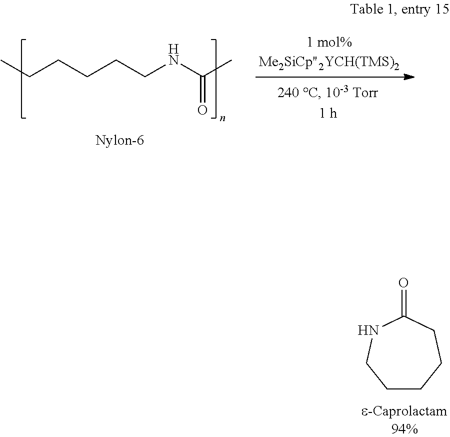

Exactly 98.8 mg of Nylon-6 pristine powder (0.89 mmol) and 5.3 mg of Me₂SiCp″₂YCH(TMS)₂ (8.9 μmol, 1 mol %) were reacted according to the general procedure, heating the reaction mixture to 240° C. for 1 h. Exactly 17.6 mg of mesitylene (0.146 mmol) was used as an internal standard. ¿-caprolactam was obtained in 94% yield.

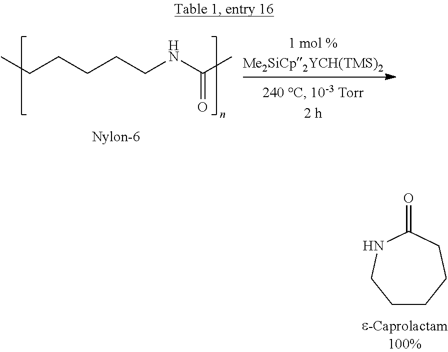

Exactly 99.8 mg of Nylon-6 pristine powder (0.89 mmol) and 5.3 mg of Me₂SiCp″₂YCH(TMS)₂ (8.9 μmol, 1 mol %) were reacted according to the general procedure, heating the reaction mixture to 240° C. for 2 h. Exactly 14.3 mg of mesitylene (0.119 mmol) was used as an internal standard. ε-caprolactam was obtained in 100% yield.

### Procedures and NMR Spectra for Reactions in Table 2

For the NMR spectra see the General Note above with respect to Table 1.

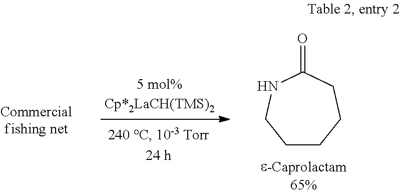

Exactly 100.3 mg of commercial fishing net (0.89 mmol) and 27.5 mg of Cp*₂LaCH(TMS)₂ (45 μmol, 5 mol %) were reacted according to the general procedure, heating the reaction mixture to 240° C. for 24 h. During the reaction, the solid mixture at the bottom of the reaction tube turned dark red and eventually to a dark black color. Exactly 15.9 mg of mesitylene (0.132 mmol) was used as an internal standard. ¿-caprolactam was obtained in 65% yield.

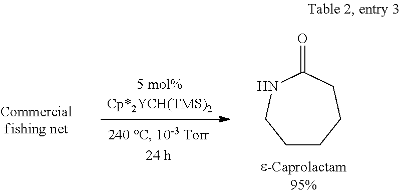

Exactly 98.0 mg of commercial fishing net (0.89 mmol) and 24.3 mg of Cp*₂YCH(TMS)₂ (45 μmol, 5 mol %) were reacted according to the general procedure, heating the reaction mixture to 240° C. for 24 h. During the reaction, the color of the solid mixture at the bottom of the reaction tube had no major changes. Exactly 20.6 mg of mesitylene (0.171 mmol) was used as an internal standard. ε-caprolactam was obtained in 95% yield.

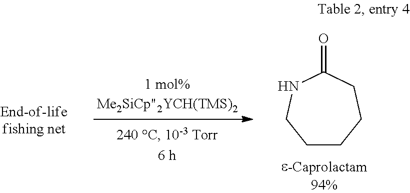

Exactly 93.0 mg of end-of-life fishing net (0.82 mmol) and 5.0 mg of Me₂SiCp″₂YCH(TMS)₂ (8.4 μmol, 1 mol %) were reacted according to the general procedure, heating the reaction mixture to 240° C. for 6 h. Exactly 30.6 mg of mesitylene (0.255 mmol) was used as an internal standard. ¿-caprolactam was obtained in 94% yield.

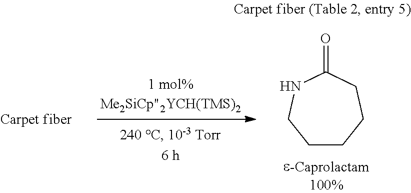

Exactly 106.0 mg of carpet fiber (0.94 mmol) and 5.4 mg of Me₂SiCp″₂YCH(TMS)₂ (9.1 μmol, 1 mol %) were reacted according to the general procedure, heating the reaction mixture to 240° C. for 6 h. Exactly 29.8 mg of mesitylene (0.248 mmol) was used as an internal standard. ε-caprolactam was obtained in 100% yield.

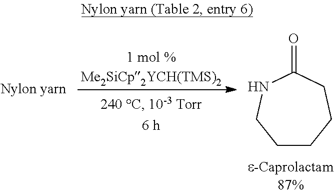

Exactly 100.0 mg of carpet fiber (0.89 mmol) and 5.3 mg of Me₂SiCp″₂YCH(TMS)₂ (8.9 μmol, 1 mol %) were reacted according to the general procedure, heating the reaction mixture to 240° C. for 6 h. Exactly 27.6 mg of mesitylene (0.230 mmol) was used as an internal standard. ε-caprolactam was obtained in 87% yield.

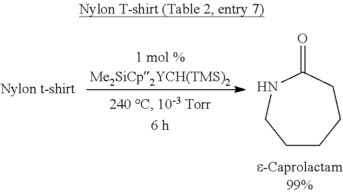

Exactly 101.2 mg of Nylon T-shirt (0.89 mmol) and 5.3 mg of Me₂SiCp″₂YCH(TMS)₂ (8.9 μmol, 1 mol %) were reacted according to the general procedure, heating the reaction mixture to 240° C. for 6 h. Exactly 18.3 mg of mesitylene (0.152 mmol) was used as an internal standard. ¿-caprolactam was obtained in 99% yield.

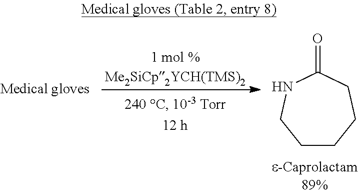

Exactly 355 mg of medical gloves [containing 31% of Nylon-6 (100 mg, 0.89 mmol), see “General methods and materials” for details) and 5.3 mg of Me₂SiCp″₂YCH(TMS)₂ (8.9 μmol, 1 mol %) were reacted according to the general procedure, heating the reaction mixture to 240° C. for 12 h. Exactly 17.4 mg of mesitylene (0.145 mmol) was used as an internal standard. ¿-caprolactam was obtained in 89% yield (with regards to the Nylon-6 content in the medical gloves).

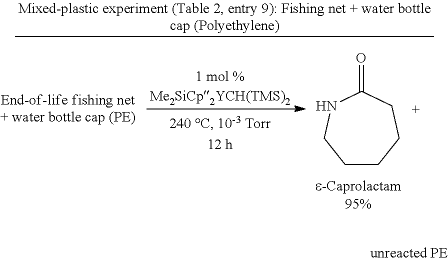

Exactly 101.2 mg of end-of-life fishing net (0.89 mmol), 100 mg of chopped water bottle cap (polyethylene) and 5.3 mg of Me₂SiCp″₂YCH(TMS)₂ (8.9 μmol, 1 mol %) were reacted according to the general procedure, heating the reaction mixture to 240° C. for 12 h. Exactly 26.1 mg of mesitylene (0.217 mmol) was used as an internal standard. ε-caprolactam was obtained in 95% yield (with regards to the amount of the end-of-life fishing net added). The solid residual (catalyst+PE) was separated from the reaction mixture, washed with acetone multiple times and dried under high vacuum. Next, 2 mL of C₂Cl₄D₂ (TCE-d₂) was then added to the solid residue and heated at 150° C. until most of the PE dissolved. Then, 0.5 mL of the filtrate was charged in NMR tube. 1H-NMR analysis was performed for the residual plastic, which confirmed that the water bottle cap (PE) was unreacted.

### Results and Discussion

### Organolanthanide Catalyst Design

Commercial virgin Nylon-6 powder was first utilized to screen catalysts and catalytic conditions, to minimize/exclude the potential influence on depolymerization results reflecting poor polymer-catalyst contact, uneven mixing/heating, and plastic additives. The molecular weight (Mₙ) of the virgin Nylon-6 powder was determined to be 11,930 g mol⁻¹ by Gel Permeation Chromatography (GPC). All depolymerizations were performed in a solventless mode, with crystalline ε-caprolactam collecting on the reactor cold wall (See FIG. 2A).

In initial attempts to lower the catalyst loading of 1 from 5 mol % to 1 mol %, a moderate ε-caprolactam yield was obtained (69%, Table 1, entry 1) in 0.5 h reaction time at 240° C. Further decreasing the catalyst loading of 1 to 0.2 mol % depressed the depolymerization yield to 5.6% (Table 1, entry 2), which prompted catalyst substitution. Without wishing to be bound to a particular theory, it was hypothesized that, after depolymerization is initiated, the three reactive La-amide linkages may promote macromolecule crosslinking and catalyst immobilization (see below for detailed mechanistic studies).

Based on the inventors' insights, the organolanthanide coordination sphere was modified using non-dissociable ancillary x-ligands to sterically shield the highly electrophilic lanthanide centers while reducing impediments introduced by multiple reaction linkages. Lanthanocenes bearing a variety of non-dissociable supporting x-ligands such as bis(pentamethylcyclopentadienyl) (Cp*₂) were selected. The La coordination sphere was first modified by replacing two -TMS₂N active sites with ancillary Cp* ligands (Cp*₂LaN(TMS)₂, 2). Reflecting the inherent unpredictability in organometallic chemistry, however, this modification led to a reduced depolymerization activity even with 1 mol % catalyst and an extended reaction time (17% in 6.0 h; Table 1, entry 3). It was thought that this may reflect the reduced number of La-NTMS₂ active sites and the introduction of sterically demanding ligands (Cp*₂). However, additional insights by the inventors led to use of an alternative active site to compensate for aforementioned deficiencies and increase the depolymerization efficiency.

Thus, next, Cp*₂LaCH(TMS)₂ (3) was investigated for Nylon-6 depolymerization. Strikingly, catalyst 3 was found to be a highly effective catalyst, at 1 mol % loading providing a 91% yield in only 0.5 h of reaction time at 240° C. (Table 1, entry 4). This result represents a significant advance over catalyst 2, demonstrating a key role for increasing the basicity of the sigma-bonded La ligand (from —N(TMS)₂ to —CH(TMS)₂) in initiating the catalytic cycle, in good agreement with DFT calculations (vide infra). Extending the reaction time from 0.5 h to 1 h afforded an excellent yield of 99% for the 3-mediated depolymerization (Table 1, entry 5). Additionally, 3 was found to be very active even with a lower catalyst loading of 0.2 mol % to provide a high yield of 93% (Table 1, entries 6,7), in marked contrast to catalyst 1 (5.6% yield). To examine the scalability of this reaction, the 3-mediated depolymerization was carried out on a gram scale using a 0.2 mol % catalyst loading. An NMR yield of 97% (94% isolated yield) was obtained (Table 1, entry 8).

TABLE 1
Optimization of the catalysts and conditions for catalytic
Nylon-6 depolymerization shown in FIG. 2A.^{a}

| | | Catalyst | | | |
|---|---|---|---|---|---|
| | | loading | Time | Temperature | Yield |
| Entry | Catalyst | (mol %) | (h) | (°C) | (%)^{b} |
| 1 | 1 | 1.0 | 0.5 | 240 | 69 |
| 2 | 1 | 0.2 | 1.0 | 240 | 5.6 |
| 3 | 2 | 1.0 | 6.0 | 240 | 17 |
| 4 | 3 | 1.0 | 0.5 | 240 | 91 |
| 5 | 3 | 1.0 | 1.0 | 240 | 99 |
| 6 | 3 | 0.2 | 1.0 | 240 | 93 |
| 7 | 3 | 0.2 | 0.5 | 240 | 81 |
| 8 ^{c} | 3 | 0.2 | 3.0 | 240 | 97 (94)^{d} |
| 9 | 3 | 1.0 | 2.0 | 220 | 90 |
| 10 | 3 | 0.2 | 4.0 | 220 | 95 |
| 11 | 1 | 1.0 | 2.0 | 220 | 0 |
| 12 | 4 | 1.0 | 1.0 | 240 | 24 |
| 13 | 5 | 1.0 | 1.0 | 240 | 2.2 |
| 14 | 5 | 5.0 | 24 | 240 | 93 |
| 15 | 6 | 1.0 | 1.0 | 240 | 94 |
| 16 | 6 | 1.0 | 2.0 | 240 | 100 |
| 17 | 7 | 1.0 | 0.5 | 240 | 91 |
| 18^{e} | 7 | 1.0 | 2.0 | 240 | 95^{e} |

^{a}General depolymerization conditions: static vacuum (10⁻³ Torr), 100 mg of Nylon-6 powder in 50 mL Schlenk flask, unless otherwise stated.
^{b}Yields determined by ¹H NMR with a mesitylene internal standard.
^{c} Scaled-up depolymerization: 1.0 g of Nylon-6 in 100 mL Schlenk flask.
^{d}Isolated yield after recrystallization of product ε-caprolactam from hexane/acetone; see “General methods and materials” for details.
^{e}Nylon-4 was used, and the yield was calculated based on its monomer. 2-pyrrolidinone.

Another consideration is the depolymerization reaction temperature. Chemical recycling of commodity polymers typically utilizes high reaction temperatures and therefore, is energy-intensive and may be accompanied by undesired thermal decomposition. Although the present 240° C. reaction temperature is lower than used in existing techniques for Nylon-6 depolymerization to ε-caprolactam (>300° C.), it is above the Nylon-6 (218.3° C.) melting point. In an effort to lower the reaction temperature, catalytic studies were next conducted at 220° C. with 1 mol % catalyst 3 loading for 2 h with a respectable yield (Table 1, entry 9) and 95% yield for 4 h and 0.2 mol % catalyst loading (95% yield; Table 1, entry 10). Note that this temperature is, to the inventors' knowledge, the lowest reported Nylon-6 depolymerization temperature under solvent-free condition. In comparison, catalyst 1 was completely inactive at 220° C. (Table 1, entry 11).

### Metal Ion Size and Ancillary Ligand Effects

To explore any correlations between catalytic activity and Ln³⁺ radius, the Cp*₂LnCH(TMS)₂ homologs (FIG. 2B) where Ln=Lu (4; ionic radius (i.r.)=0.97 Å)=1.01 5; i.r.=1.01 Å) were prepared to compare with Ln=La (3; i.r.=1.17 Å) Nylon-6 depolymerization characteristics. With 1 mol % loading, the Lu³⁺ catalyst affords lower ε-caprolactam yields (24%, Table 1, entry 12) in 1 h, while catalyst 5, yields only 2.2% ε-caprolactam under identical reaction conditions (Table 1, entry 13), both substantially lower than that of catalyst 3 (99%; Table 1, entry 4). Achieving 93% yield of ε-caprolactam is possible with catalyst 5, however, this requires a 5 mol % loading and 24 h (Table 1, entry 14). These results qualitatively parallel activity trends for Cp*2Ln-catalyzed olefin hydroelementation and other additions to C—C unsaturation, which is surprising as these reactions involve a mix of electronic and steric effects, with the latter predominant and reflecting crowding in the olefin insertion transition state for hydroamination in which olefin is inserted into a lanthanide-nitrogen bond.

Nevertheless, smaller ionic radius Y³⁺ metallocene complexes can display higher thermal stabilities. Y also lies at the lower end of the energy, cost, and environmental impact scale, and also displays significantly lower toxicity, reflected in a significantly higher median effect dose concentration (EC₅₀) of 6.7 μg/L vs that of La (EC₅₀=147 μg/L). Thus, even more effective Y catalysts were explored by further modifying the Y coordination sphere.

Specifically, chelating ansa-bridged ligation, e.g., Me₂SiCp″₂Ln-(Cp″=η⁵-Me₄C₅) (see FIG. 2B) was explored for larger “bite” angles and enhanced thermal robustness. Specifically, Me₂SiCp″₂YCH(TMS)₂ (6) was prepared and examined for the virgin Nylon-6 powder depolymerization. Remarkably, “opening” of the Y coordination sphere dramatically increases the depolymerization yield from 2% to 94% (Cp*₂Y-→Me₂SiCp″₂Y-) under identical reaction conditions (Table 1, entry 13 vs 15), illustrating the surprising effects of this ancillary ligand “engineering.” Further extending the reaction time to 2 h effects a 100% conversion of Nylon-6 by catalyst 6 (Table 1, entry 16). This ligand manipulation essentially imbues a more desirable Y center with La center catalytic properties.

The inventors' insights further led to incorporating a metal center with a larger Ln³⁺ radius such as Sm^{3+ on the basis that the coordination sphere of the ansa-structure may be able to be further maximized. Thus, Me}₂SiCp″₂SmCH(TMS)₂ (7) was next prepared and tested for Nylon-6 depolymerization. The catalyst showed an improved rate and achieved 91% yield of caprolactam in shorter reaction time (30 min; Table 1, entry 17). Finally, the scope of polyamides for ring closing depolymerization was examined using optimal ansa-metallocene catalyst 7. Nylon-4 was successfully depolymerized to 2-pyrrolidinone with 95% yield at 240° C. (Table 1, entry 18).

### Depolymerization of Post-Consumer Nylons

Encouraged by the high activities of lanthanocene catalysts 3 and 6 for finely powdered Nylon-6, the chemical recycling of “real-world” post-consumer Nylon-6 products was next examined (Table 2). These samples were manually chopped into smaller pieces, dried, and loaded into the aforementioned reaction flasks for depolymerization experiments (see “General methods and materials” for details). With relevance to ocean pollution, an end-of-life fishing net, used for 2 years, was first investigated. Beginning with catalyst 1, which was found to rapidly sublime from the catalyst+finely chopped fishing net mixture, the yield of ε-caprolactam was negligible (Table 2, entry 1) after 24 h/240° C. For catalyst 3, the modest yield at high loadings observed (Table 2, entry 2) likely reflects poor mixing and the known thermal instability of this catalyst, while thermally more robust Y catalyst 5 delivers slightly higher yields (Table 2, entry 3), and coordinatively more open ansa-metallocene Y catalyst 6 achieves a remarkable 94% yield in only 6 h (Table 2, entry 4), paralleling the results with pristine powdered Nylon-6, likely reflecting enhanced activity and thermal robustness.

To investigate the scope of 6-catalyzed depolymerizations, other post-consumer Nylons were next examined. Carpet waste poses a global environmental impact. With an annual carpet production of 12 billion feet only 5% is recycled while 91% ends up in landfills; in the U.S. alone, 4 billion lb of carpet are discarded annually in landfills. With an estimated that 75% of carpets composed of Nylon, regenerating ε-caprolactam from carpet waste would be ideal. Pleasingly, a sample of carpet fiber was fully depolymerized with 1 mol % of 6, providing a quantitative yield of ε-caprolactam (Table 2, entry 5). Next examined was post-consumer Nylon-6 yarn, which yielded 87% ε-caprolactam (Table 2, entry 6) under the same reaction conditions. Nylon fabrics, known for their durability and water resistance, are widely utilized textiles to manufacture shirts, swimsuits, footwear, etc. A Nylon t-shirt was next cut into small pieces and subjected to depolymerization using catalyst 6, producing ε-caprolactam with an impressive 99% yield (Table 2, entry 7).

TABLE 2
Catalytic depolymerization of post-consumer Nylon plastics.^{a}

| | | | Catalyst | | |
|---|---|---|---|---|---|
| | | | loading | Time | Yields |
| Entry | Nylon plastic | Catalyst | (mol %) | (h) | (%)^{b} |
| 1 | Fishing net | 1 | 5.0 | 24 | 0 |
| 2 | Fishing net | 3 | 5.0 | 24 | 65 |
| 3 | Fishing net | 5 | 5.0 | 24 | 95 |
| 4 | Fishing net | 6 | 1.0 | 6.0 | 94 |
| 5 | Carpet fiber | 6 | 1.0 | 6.0 | 100 |
| 6 | Nylon yarn | 6 | 1.0 | 6.0 | 87 |
| 7 | T-shirt | 6 | 1.0 | 6.0 | 99 |
| 8 | Medical gloves ^{c} | 6 | 1.0 | 12 | 89^{d} |
| 9 | Fishing net + | 6 | 1.0 | 12 | 95 |
| | water bottle cap^{e} | | | | |

^{a}General reaction conditions: static vacuum (10⁻³ Torr), 100 mg scissor-chopped end-of-life Nylon in 50 mL Schlenk flask.
^{b}Yields determined by ¹H NMR with mesitylene internal standard.
^{c} 355 mg sample of medical gloves, which contain 100 mg Nylon-6 (31%).
^{d}Determined from Nylon-6 content in medical glove sample; see SI for details.
^{e}Fishing net:water bottle cap in 1:1 mass ratio.

To expand the scope of this sustainability study, the focus was extended to pandemic-associated plastic pollution resulting from the enormous demand for medical plastics such as face masks and gloves to combat COVID-19. A pair of Nylon medical gloves containing 31% of Nylon-6 was cut and subjected to polymer deconstruction with catalyst 6 (Table 2, entry 8). The 6-mediated depolymerization of medical gloves furnished 89% yield of ε-caprolactam with respect to the Nylon content (see “General methods and materials” for details). Finally, in real-world marine plastic wastes, fishing net debris is typically mixed with other plastics such as containers, bags, straws, bottle caps, etc. Besides Nylon wastes, polyolefins such as polyethylene and polypropylene (PE, PP) are the most common polymers in ocean plastics. To investigate the applicability of catalyst 6 to post-consumer plastic mixtures, depolymerization of end-of-life Nylon-6 fishing net was carried out admixed with a PE water bottle cap in a 1:1 mass ratio. This afforded ε-caprolactam in a 95% yield (Table 2, entry 9), while the unreactive PE bottle cap was recovered unchanged, as evidenced by 1H NMR analysis. This result indicated that catalyst 6 is compatible with mixed-plastic scenarios and can be utilized for the separation of end-of-life Nylon-6 from mixed-plastic, yielding the valuable caprolactam monomer.

The experiments described above demonstrate the potential for Nylon-6 depolymerization with ultra-low catalyst loadings in a continuous rather than batch process. To validate this approach, in additional experiments, the loading of catalyst 3 was reduced from 0.2 mol % to 0.1 mol % along with introducing additional fresh Nylon-6 before reaching full conversion of the first batch. To further probe the recyclability of the present metallocene catalysts, a multi-batch simulated continuous depolymerization experiment was performed in a larger-scale reactor with a 0.04 mol % loading of catalyst 3. Before each run (runs 2-5), the caprolactam collected from the previous run was removed, weighed, and 250 mg of fresh Nylon-6 was added to the reactor containing the same catalyst 3 charge. The results showed steady caprolactam yields across all catalyst recycling experiments, indicating that catalyst 3 retains a high level of activity through each run. The final (6th) run was performed without fresh Nylon addition to allow full completion of the depolymerization process, providing an overall Nylon-6 yield of 95.5% using an ultra-low overall catalyst loading of 0.04 mol %.

### CONCLUSIONS

This Example demonstrates a surprising pathway to realize efficient and practical Nylon plastics recycling. By fine-tuning the catalytic structures, a series of lanthanocenes were investigated, leading to the discovery that many are surprisingly efficient catalysts for the chemical recycling of Nylon-6 in a solventless, scalable process. For example, catalyst 3 is a highly effective catalyst for the depolymerization of powdered Nylon-6 (affording up to 100% yield of the monomer-caprolactam) at temperatures as low as 220° C. and catalyst loadings as low as 0.2 mol %. These conditions, to the best of the inventors' knowledge, are the mildest to date for Nylon-6 depolymerization to ε-caprolactam. For the more challenging deconstruction of waste Nylon-6 commodity products, ansa-metallocene complex 6 was found to efficiently depolymerize a variety of end-of-life Nylon plastics, including fishing nets, carpets, clothing, gloves, as well as in a mixed-plastic case. Moreover, the ε-caprolactam collected from these end-of-life Nylons demonstrated to be re-polymerizable to pristine Nylon-6, “closing the loop” and achieving model circularity for Nylon plastics. Mechanistic experiments show that the implementation of non-dissociable x-ancillary ligands in these effective catalysts is crucial in sterically shielding the metal center to allow intrachain “unzipping” and effective interchain “hopping” mechanisms, minimizing catalyst cross-linkages and chain-end deactivations.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A method for depolymerizing a polyamide, the method comprising combining a polyamide and an organolanthanide catalyst to depolymerize the polyamide to a product, wherein the organolanthanide catalyst comprises a lanthanide metal, an active ligand bound to the lanthanide metal, and an ancillary ligand bound to the lanthanide metal, wherein the ancillary ligand is selected from conjugated carbocyclic groups and the active ligand is selected from those having a formula -EHₘ(XRₙ)ₒ,
wherein E is selected from C, N, P, Si, Ge, and Sn;
X is independently selected from Si, H, N, P, C, Ge, and Sn; and
R is independently selected from alkyl, H, aryl, alkoxyl, silyl, germyl, and stannyl;
wherein m is 1 or 0, n is 3 or 2, and o is 2 or 3.

2. The method of claim 1, wherein E is selected from C, Si, Ge, and Sn.

3. The method of claim 1, wherein the organolanthanide catalyst comprises a single active ligand and two ancillary ligands.

4. The method of claim 1, wherein the ancillary ligand is selected from unsubstituted, monocyclic conjugated carbocyclic groups.

5. The method of claim 4, wherein the ancillary ligand is selected from pentamethylcyclopentadienyl and cyclopentadienyl.

6. The method of claim 1, wherein the organolanthanide catalyst comprises a single active ligand and two ancillary ligands connected via a bridging group selected from silyl groups and alkyl groups.

7. The method of claim 1, wherein E is selected from C, Si, Ge, and Sn, m=1, and o=2.

8. The method of claim 7, wherein E is C, X is Si, and n=3.

9. The method of claim 8, wherein R is alkyl or H.

10. The method of claim 1, wherein the organolanthanide catalyst comprises a single active ligand and two ancillary ligands selected from unsubstituted, monocyclic conjugated carbocyclic groups, and wherein E is C, X is Si, R is alkyl or H, m=1, n=3, and o=2.

11. The method of claim 10, wherein the lanthanide metal is selected from La, Y, or SmY, La, or Sm.

12. The method of claim 11, wherein the two ancillary ligands are connected via a bridging group selected from alkyl groups and silyl groups.

13. The method of claim 1, wherein the organolanthanide catalyst is Me₂Si(η⁵—C₅Me₄)₂LnCH(SiMe₃)₂ or (η⁵—C₅Me₅)₂LnCH(SiMe₃)₂.

14. The method of claim 13, wherein Ln is Y, La, or Sm.

15. (canceled)

16. The method of claim 1, wherein the polyamide is selected from poly(hexano-6-lactam) (Nylon-6), poly(2-pyrrolidinone) (Nylon-4), poly(2-piperidone) (Nylon-5), polyenanthamide (Nylon-7), polycapryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), poly[imino (1,6-dioxo hexamethylene)imino hexamethylene] (Nylon-66), and combinations thereof.

17. The method of claim 1, wherein the product comprises a monomer from which the polyamide was formed.

18. The method of claim 17, wherein the monomer is a cyclic amide.

19. (canceled)

20. The method of claim 1, wherein the method is carried out using a temperature of 250° C. or less, a loading of the organolanthanide catalyst of 5 mol % or less, a time of 24 hours or less, and the method provides a yield of the product of at least 90%.

21. (canceled)

22. The method of claim 1, wherein the method is carried out using a continuous flow reactor system.

23. (canceled)

24. The method of claim 1, wherein the organolanthanide catalyst is Me₂Si(η⁵—C₅Me₄)₂LnCH(SiMe₃)₂ or (η⁵—C₅Me₅)₂LnCH(SiMe₃)₂, and further wherein the method is carried out using a temperature of 250° C. or less and a loading of the organolanthanide catalyst of 5 mol % or less.

25. The method of claim 24, wherein Ln is Y, La, or Sm.

26. The method of claim 25, wherein the polyamide is poly(hexano-6-lactam) (Nylon-6), poly[imino (1,6-dioxo hexamethylene)imino hexamethylene] (Nylon-66), or a combination thereof.
